(19) United States
(12) Patent Application Publication
(10) Pub. No.: US 2012/0055272 A1
(43) Pub. Date: Mar. 8, 2012

(54) PAPER ROLL DETECTION APPARATUS AND METHOD, AND PRINTER

(75) Inventors: Tsuyoshi Sanada, Singapore (SG); Toshiharu Sekino, Shizuoka (JP)

(73) Assignee: TOSHIBA TEC KABUSHIKI KAISHA, Tokyo (JP)

(21) Appl. No.: 13/190,777

(22) Filed: Jul. 26, 2011

(30) Foreign Application Priority Data
Sep. 3, 2010 (JP) 2010-198193

(51) Int. Cl. G01N 33/34

(52) U.S. Cl. 73/865.8

(57) ABSTRACT

Embodiments described herein are to an apparatus for detecting an amount of paper in a roll of paper, which includes a container including a support surface, and first and second recessed portions connected to each other on the support surface, the roll of paper being contained in one of the first and second recessed portions. The apparatus further includes a first sensor to detect whether an outer periphery of the roll of paper contained in one of the first and second recessed portions reaches a first outer diameter, and a second sensor to detect whether the outer periphery of the roll of paper contained in one of the first and second recessed portions reaches a second outer diameter smaller than the first outer diameter. The apparatus further includes an estimation unit to estimate a remaining amount of the paper based on a thickness of the paper.

### CROSS-REFERENCE TO RELATED APPLICATION

This application is based upon and claims the benefit of priority from Japanese Patent Application No. 2010-198193, filed on Sep. 3, 2010, the entire contents of which are incorporated herein by reference.

### FIELD

Embodiments described herein relate generally to a paper roll detection apparatus and a method thereof, and a printer that estimates the remaining amount of paper in a roll of paper.

### BACKGROUND

An apparatus which detects the remaining amount of paper in a roll of paper may be used in, for example, a point-of-sale (“POS”) printer. The position of a printer employing such an apparatus may be selectively adjusted. For example, the printer may be mounted on a desk or a wall.

Such a printer includes a hopper to contain a roll of paper therein. The hopper may include a first recessed portion and a second recessed portion. The first recessed portion may be utilized to contain a remaining portion of the roll of paper when the printer is mounted on a horizontal surface such as a desk (e.g., in a desk mounting mode). On the other hand, the second recessed portion may be utilized to contain the remaining portion of the roll of paper when the printer is mounted on a vertical surface such as a wall (e.g., in a wall mounting mode).

As subsequent printing jobs proceed, the remaining amount of paper in the roll of paper contained in the first or the second recessed portion gradually decreases. When a detection unit of the printer determines that the remaining amount of paper in the roll of paper is equal to or less than a predetermined level, the printer may output a message prompting a user to replace the current remaining roll of paper with a new one.

The aforementioned printer is configured to determine the remaining amount of paper in the roll of paper without detecting the thickness of the remaining roll of paper. However, this detection method causes a large margin of error in detecting the remaining amount of paper in the roll of paper depending on the type of paper being used. As a result, the paper may run out before the roll of paper can be replaced with a new one, or otherwise, an excessive amount of paper in the remaining roll of paper may be unnecessarily replaced with a new one.

### BRIEF DESCRIPTION OF THE DRAWINGS

FIG. 1 is a side elevational view of a printer mounted in a desk mounting mode in accordance with one illustrative embodiment.

FIG. 2 is a side sectional view of a printer mounted in a wall mounting mode in accordance with of one illustrative embodiment.

FIG. 3 is a side elevational view showing a state in which the remaining amount of paper in the roll of paper is detected in accordance with one illustrative embodiment.

FIG. 4 is a side elevational view showing a state in which the remaining amount of paper in the roll of paper is detected in accordance with another illustrative embodiment.

FIG. 5 is a side elevational view showing a state in which the remaining amount of paper in the roll of paper is detected in accordance with still another illustrative embodiment.

FIG. 6 is a side elevational view showing a state in which the remaining amount of paper in the roll of paper is detected in accordance with still another illustrative embodiment.

FIG. 7 is a block diagram showing a configuration of a printer according to one illustrative embodiment.

### DETAILED DESCRIPTION

According to one embodiment, an apparatus for detecting an amount of paper in a roll of paper, the paper being wound around a core of the roll of paper, which includes a container including a support surface, and first and second recessed portions connected to each other on the support surface, wherein the roll of paper can be contained in one of the first and second recessed portions. The apparatus further includes a first sensor configured to detect whether an outer periphery of the roll of paper contained in one of the first and second recessed portions reaches a first outer diameter, and a second sensor configured to detect whether the outer periphery of the roll of paper contained in one of the first and second recessed portions reaches a second outer diameter smaller than the first outer diameter. The apparatus further includes an estimation unit configured to estimate a remaining amount of the paper based on a thickness of the paper, wherein the thickness of the paper is estimated based on a length of paper discharged during a time interval between a point of time when the first sensor detects the outer periphery of the roll of paper at the first outer diameter and a point of time when the second sensor detects the outer periphery of the roll of paper at the second outer diameter.

Embodiments will now be described in detail with reference to the drawings.

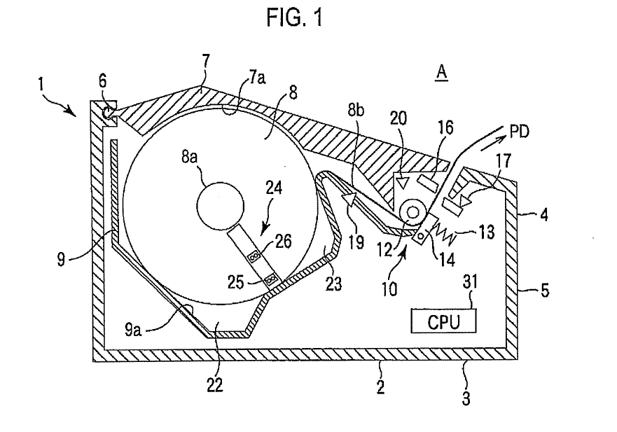

FIG. 1 is a side elevational view of a printer according to one illustrative embodiment.

A printer A, which prints information on paper wound in a roll, includes a printer main body 1 which may be horizontally mounted in a widthwise direction (e.g., on a desk) or vertically mounted in a lengthwise direction (e.g., on a wall). In FIG. 1, the printer main body 1 is shown to be horizontally mounted, for example, on a desk.

As shown in FIG. 1, in the printer A, a wide-side surface 2 of the printer main body 1 corresponds to a first mounting portion 3 configured to mount the printer main body 1 in a desk mounting mode. On the other hand, a narrow-side surface 4 corresponds to a second mounting portion 5 configured to mount the printer main body 1 in a wall mounting mode. Further, a cover 7 is installed in the printer main body 1 such that the cover 7 may open or close the printer main body 1 by rotating around a hinge 6.

The printer main body 1 further includes a hopper 9 (serving as a roll of paper container to accommodate a roll of paper 8 therein) and a print mechanism 10. The roll of paper 8 is formed by winding paper 8b around a core 8a in a roll. The cover 7 includes an arc-shaped recess 7a that is formed to have a certain gap between the cover 7 and the roll of paper 8 even when the paper 8b is wound around the core so that the roll of paper has its largest diameter.

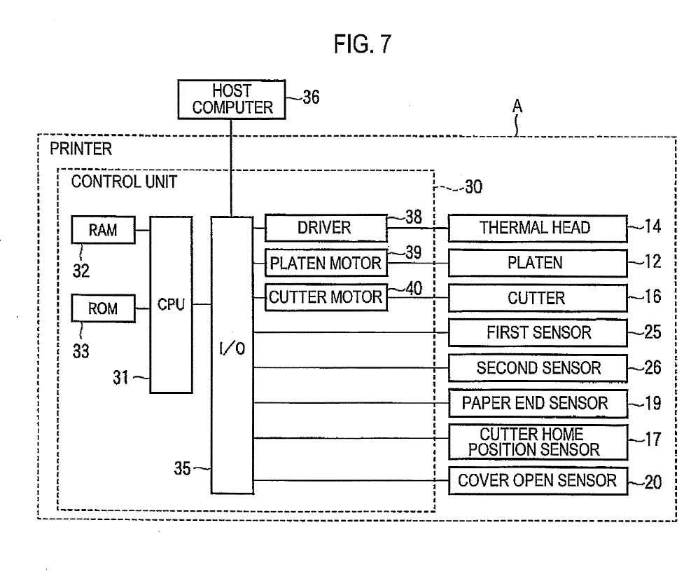

The print mechanism 10 includes a platen 12 configured to be rotated by a platen motor 39 (see FIG. 7). In one embodiment, the platen motor 39 may be a step motor configured to generate driving pulses to drive the rotation of the platen 12. The print mechanism 10 further includes a thermal head 14 configured to print information on the paper 8b interposed between the platen 12 and the thermal head 14, which is urged against the platen 12 by means of a head spring 13.

Disposed downstream from the print mechanism 10 in a paper discharge direction PD are a cutter 16 configured to be rotated by a cutter motor 40 (see FIG. 7) and a cutter home position sensor 17 configured to detect a home position where the cutter 16 is repositioned after a cutting operation.

A paper end sensor 19, which is disposed between the hopper 9 and the platen 12 upstream of the print mechanism 10 in the paper discharge direction PD, detects the presence or absence of the paper 8b passing over the sensor.

A cover open sensor 20, which is disposed above the platen 12, detects the opening or closing of the cover 7.

The hopper 9 as described above includes a support surface 9a to support the outer periphery of the roll of paper 8.

As shown in FIG. 1, either in case the printer main body 1 is horizontally mounted with the first mounting portion 3 facing downward or in case the printer main body 1 is vertically mounted with the second mounting portion 5 facing downward, the outer periphery of the roll of paper 8 is biased vertically toward the support surface 9a due to its own weight. In this way, the support surface 9a can support the roll of paper 8 in any circumstance.

In one embodiment, the support surface 9a includes a first recessed portion 22 and a second recessed portion 23, which are coupled to each other and spaced apart by a certain distance on the support surface 9a. The first recessed portion 22 lies on a virtual vertical line passing through the center of the roll of paper 8 in case the printer main body 1 is horizontally mounted with the first mounting portion 3 facing downward. On the other hand, the second recessed portion 23 lies on a virtual vertical line passing through the center of the roll of paper 8 in case the printer main body 1 is vertically mounted with the second mounting portion 5 facing downward.

In some embodiments, the size of the first and second recessed portions 22 and 23 may be predetermined so that the respective portions stably accommodate therein the roll of paper 8, which hangs down due to its own weight, when the outer periphery of the roll of paper 8 is equal to or less than a predetermined value as subsequent printing job proceeds.

Meanwhile, a roll of paper detecting device 24 is disposed at one side of the hopper 9 in a vertical direction with respect to the plane view of FIG. 1.

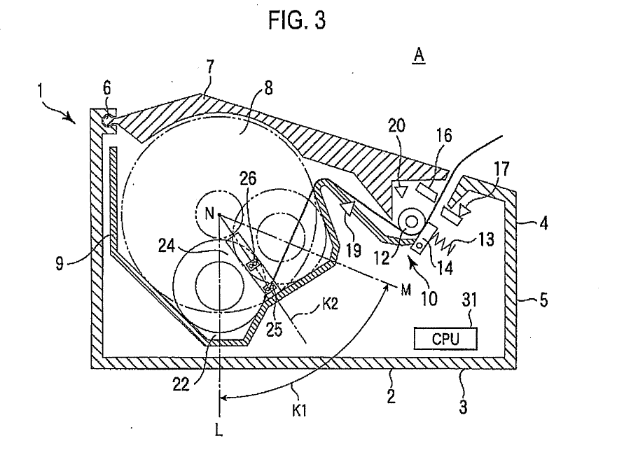

FIG. 3 is a side elevational view showing a state in which the remaining amount of paper in the roll of paper 8 is detected in accordance with one illustrative embodiment. In FIG. 3, it is assumed that N indicates a virtual center of the roll of paper 8 with its greatest winding diameter (i.e., maximum outer diameter). Also, N-L indicates a line defined by connecting between the center N and the center of the roll of paper 8 when it is contained in the first recessed portion 22, while N-M indicates a line defined by connecting between the center N and the center of the roll paper 8 when it is contained in the second recessed portion 23. Under this assumption, an angle K1 formed between the lines N-L and N-M is set to be less than 90 degrees.

Further, the roll of paper detecting device 24 includes first and second sensors 25 and 26, which are aligned in a line with respect to the center N at an angle K2, wherein the angle K2 is about half of the angle K1. While the first sensor 25 is located relatively far from the center N, the second sensor 26 is located relatively close to the center N. In one embodiment, the first sensor 25 is located farther from the center N than the second sensor 26.

The first and second sensors 25 and 26, which may be implemented by a reflective sensor using, for example, light-emitting diode (LED) and phototransistor (PHOTO) pairs, is configured to emit light to one side of the roll of paper 8 and receive light reflected therefrom, to thereby detect the presence or absence of the roll of paper 8.

FIG. 7 is a block diagram showing a configuration of a printer according to an illustrative embodiment.

As shown in FIG. 7, a printer A according to one illustrative embodiment includes a control unit 30. The control unit 30 includes a CPU 31 which is connected to a RAM 32 and a ROM 33. The control unit 30 is connected to a host computer 36 through an input/output interface (I/O) 35. The thermal head 14 is connected to the input/output interface 35 through a driver 38 configured to drive the thermal head 14. The platen 12 is connected to the input/output interface 35 through the platen motor 39 configured to drive the platen 12. The cutter 16 is connected to the input/output interface 35 through the cutter motor 40 configured to drive the cutter 16.

Further, the first sensor 25, the second sensor 26, the paper end sensor 19, the cutter home position sensor 17, and the cover open sensor 20 as described above, are connected to the input/output interface 35, respectively.

The following is a description of the operation of the roll of paper detecting device 24 as described above.

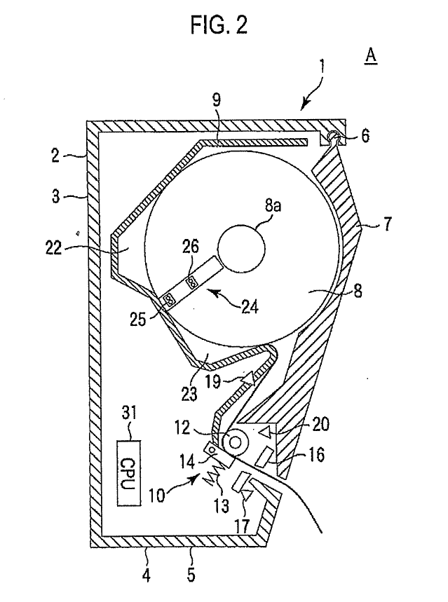

As described above, FIGS. 1 and 2 show a state where the printer A is mounted in desk mounting mode and wall mounting mode, respectively, with the roll of paper 8 with its greatest winding diameter (i.e., maximum outer diameter) contained therein. In FIGS. 1 and 2, the first and second sensors 25 and 26 determine that the roll paper 8 is loaded.

As subsequent printing jobs proceed, the outer diameter of the roll of paper 8 gradually decreases to reach a predetermined diameter. In this case, in the desk mounting mode, the roll paper 8 is contained in the first recessed portion 22 of the hopper 9. On the other hand, in the wall mounting mode, the roll paper 8 is contained in the second recessed portion 23 of the hopper 9.

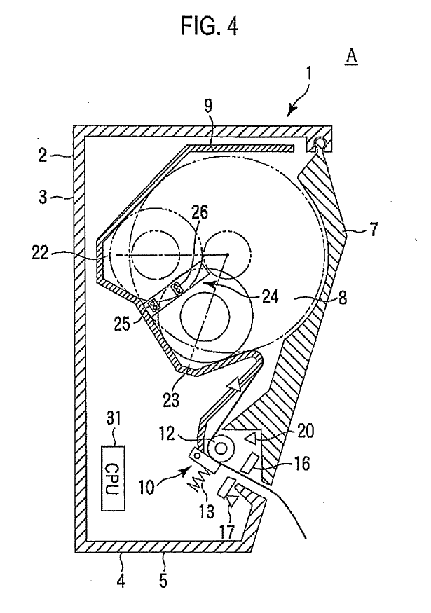

In this arrangement, if the winding diameter (i.e., diameter to the outer periphery) of the roll of paper 8 reaches a first winding diameter smaller than the predetermined diameter as further subsequent printing job proceeds, as shown in FIGS. 3 and 4, the first sensor 25 detects the absence of the roll of paper 8, while the second sensor 26 still detects the presence of the roll of paper 8 (e.g., the most outer part of the roll of paper 8).

In this case, information on a point of time at which the absence of the roll of paper 8 is first detected at the first sensor 25 is stored in the RAM 32. At the same time, the CPU 31 starts counting the number of driving pulses generated from the platen motor 39. At that point, the winding diameter (i.e., diameter to the outer periphery) of the roll of paper 8 is set to be D1.

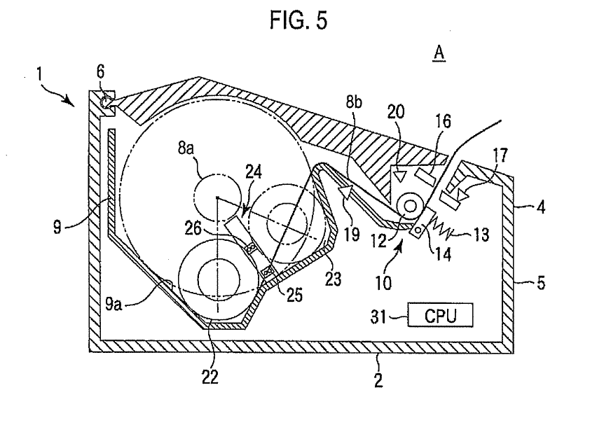

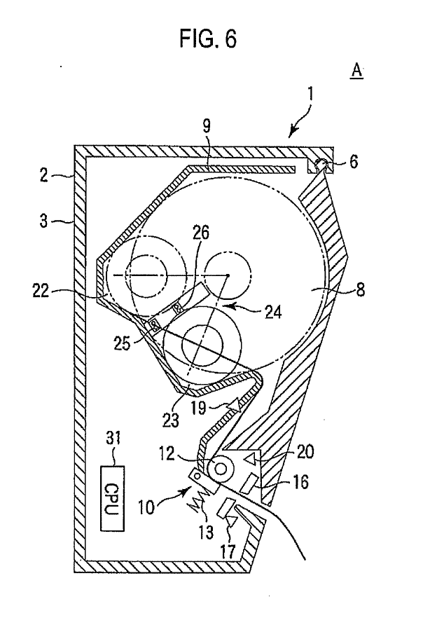

In this arrangement, if the winding diameter (i.e., diameter to the outer periphery) of the roll of paper 8 reaches a second winding diameter smaller than the first winding diameter as further subsequent printing job proceeds, as shown in FIGS. 5 and 6, the second sensor 26 as well as the first sensor 25 detects the absence of the roll of paper 8.

In this case, information on a point of time at which the absence of the roll of paper 8 is first detected at the second sensor 26 is stored in the RAM 32. At the same time, the CPU 31 stops counting the number of driving pulses generated from the platen motor 39. At that point, the winding diameter (i.e., diameter to the outer periphery) of the roll of paper 8 is set to be D2.

When the roll of paper 8 reaches its smallest winding diameter (i.e., minimum diameter to the outer periphery), only the core 8a remains in the recessed portion. The diameter of the core 8a may be predetermined and set to be D3. Alternatively, after the winding diameter D2 is determined as described above, the CPU 31 may resume counting the number of driving pulses generated by the platen motor 39 and determines whether the roll of paper 8 reaches its smallest winding diameter (i.e., minimum diameter to the outer periphery) based on the counted pulse number. If the roll of paper 8 reaches its smallest winding diameter, at that point, the winding diameter (i.e., the minimum diameter to the outer periphery) of the roll of paper 8 is set to be D3. These outer diameters D1, D2 and D3 may be values previously measured, which may be stored in the RAM 32.

The CPU 31 estimates a paper discharge length P1 based on the number of driving pulses generated by the platen motor 39, which is counted from the time when the first sensor 25 first detects the absence of the roll of paper 8 until the time when the second sensor 26 also detects the absence of the roll of paper 8. In this case, assuming that a thickness of the paper 8b is t, the following equation is established:

P1×t={(D1/2)×(D1/2)×(π)}−{(D2/2)×(D2/2)×(π)}

Thus, the paper thickness t may be derived by using the following equation:

t=[{(D1/2)×(D1/2)×(π)}−{(D2/2)×(D2/2)×(π)}]/P1 Eq. (1)

Assuming that a paper discharge length, which is discharged during a time interval between the time when the second sensor 26 first detects the absence of the roll of paper 8 (i.e., when the outer diameter of the roll of paper 8 becomes D2) and the time when the roll of paper 8 reaches its smallest winding diameter (i.e., when the minimum diameter to the outer periphery becomes D3), is P2, the following equation is established:

P2×t={(D2/2)×(D2/2)×(π)}−{(D3/2)×(D3/2)×(π)}

Thus, the paper length P2 may be derived by the following equation:

P2=[{(D2/2)×(D2/2)×(π)}−{(D3/2)×(D3/2)×(π)}]/t Eq. (2)

Information on Equations (1) and (2) as described above may be stored in the ROM 33. The CPU 31 analyzes the information stored in the ROM 33 along with the data previously stored in the RAM 32, to thereby correctly detect the remaining amount of the roll of paper 8.

This enables a user to replace a current roll of paper with a new one before the current roll of paper completely runs out. At the same time, it is possible to minimize waste of the remaining amount paper in the roll of paper.

In the above embodiments (as shown in FIG. 3), if the angle K1 between the line N-L (defined by connecting between the center N and the center of the roll of paper 8 contained in the first recessed portion 22) and the line N-M (defined by connecting between the center N and the center of the roll of paper 8 contained in the second recessed portion 23) is set to be 90 degrees, a difference between the outer diameters D1 and D2 of the roll of paper 8 becomes small, which may cause an increased margin of error in estimating the remaining amount of paper in the roll of paper. For this reason, in this embodiment, the angle K1 is set to be less than 90 degrees.

According to the above embodiments, whether the printer is installed in the desk mounting mode or in the wall mounting mode, there is no need for performing any adaptation process depending on the type of paper roll used in the printer. Also, it is not required for the printer to take into account the thickness of the paper in estimating the remaining amount of paper with a high degree of accuracy, irrespective of the printer mounting mode (e.g., the desk mounting mode and the wall mounting mode).

Therefore, by employing the above embodiments, it is possible to minimize the waste of the remaining amount of paper in the roll of paper, and on the other hand, prevent the paper from completely running out during a print job.

Furthermore, according to the above embodiments, an angle between paper guide portions (e.g., the first and second recessed portions 22 and 23), which are formed to guide the remaining roll of paper respectively in the desk mounting mode and the wall mounting mode, is set to be less than 90 degrees. Therefore, it is possible to detect the winding diameter (i.e., the outer diameter) of the roll of paper even when the diameter is relatively small, which reduces a margin of error in estimating the remaining amount of paper in the roll of paper.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An apparatus for detecting an amount of paper in a roll of paper, the paper being wound around a core of the roll of paper, the apparatus comprising:
a container including a support surface;
first and second recessed portions connected to each other on the support surface, wherein the roll of paper can be contained in one of the first and second recessed portions;
a first sensor configured to detect whether an outer periphery of the roll of paper contained in one of the first and second recessed portions reaches a first outer diameter;
a second sensor configured to detect whether the outer periphery of the roll of paper contained in one of the first and second recessed portions reaches a second outer diameter smaller than the first outer diameter; and
an estimation unit configured to estimate a remaining amount of the paper based on a thickness of the paper, wherein the thickness of the paper is estimated based on a length of paper discharged during a time interval between a point of time when the first sensor detects the outer periphery of the roll of paper at the first outer diameter and a point of time when the second sensor detects the outer periphery of the roll of paper at the second outer diameter.

2. The apparatus of claim 1, wherein the estimation unit is further configured to estimate the remaining amount of the paper by the thickness of the paper, the second outer diameter and a diameter of the core.

3. The apparatus of claim 1, wherein the length of the discharged paper is estimated based on the number of driving pulses generated by a platen motor configured to drive a platen to discharge the paper, wherein the number of driving pulses is counted from the time when the first sensor detects the outer periphery of the roll of paper at the first outer diameter until the time when the second sensor detects the outer periphery of the roll of paper at the second outer diameter.

4. The apparatus of claim 1, wherein, when N indicates the center of the roll of paper with a maximum outer diameter, a first line is defined by connecting between the center N and the center of the roll of paper when contained in the first recessed portion, and a second line is defined by connecting between the center N and the center of the roll of paper when contained in the second recessed portion, the first and second lines form a predetermined angle therebewteen.

5. The apparatus of claim 2, wherein the first and second sensors are aligned in a line with respect to the center N at about half of the predetermined angle.

6. The apparatus of claim 5, wherein the first and second sensors are spaced apart by a predetermined distance, wherein the first sensor is located farther from the center N than the second sensor.

7. The apparatus of claim 4, wherein the predetermined angle is set to be less than 90 degrees.

8. A method for detecting an amount of paper left in a roll of paper, the method comprising:
detecting, by a first sensor, whether an outer periphery of the roll of paper reaches a first outer diameter;
detecting, by a second sensor, whether the outer periphery of the roll of paper reaches a second outer diameter smaller than the first outer diameter; and
estimating, by an estimating unit, a remaining amount of the paper left in the roll of paper based on a thickness of the paper, wherein the thickness of the paper is estimated based on a length of paper discharged during a time interval between a point of time when the first sensor detects the outer periphery of the roll of paper at the first outer diameter and a point of time when the second sensor detects the outer periphery of the roll of paper at the second outer diameter.

9. The method of claim 8, wherein estimating further comprises estimating the remaining amount of the paper by the thickness of the paper, the second outer diameter and a diameter of a core.

10. The method of claim 8, wherein the length of the paper discharged is estimated based on the number of driving pulses generated by a platen motor configured to drive a platen to discharge the paper, wherein the number of driving pulses is counted from the time when the first sensor detects the outer periphery of the roll of paper at the first outer diameter until the time when the second sensor detects the outer periphery of the roll of paper at the second outer diameter.

11. The method of claim 8, wherein, when N indicates the center of the roll of paper with a maximum outer diameter, a first line is defined by connecting between the center N and the center of the roll of paper when contained in the first recessed portion, and a second line is defined by connecting between the center N and the center of the roll of paper when contained in the second recessed portion, the first and second lines form a predetermined angle therebewteen.

12. The method of claim 11, wherein the first and second sensors are aligned in a line with respect to the center N at about half of the predetermined angle.

13. The method of claim 12, wherein the first and second sensors are spaced apart by a predetermined distance, wherein the first sensor is located farther from the center N than the second sensor.

14. The method of claim 11, wherein the predetermined angle is set to be less than 90 degrees.

15. A printer for printing information on a roll of paper, the printer comprising:
a main body with a plurality of mounting portions;
a print mechanism provided in the main body;
a container provided in the main body, the container including a support surface;
first and second recessed portions connected to each other on the support surface, wherein the roll of paper can be contained in one of the first and second recessed portions;
a first sensor configured to detect whether an outer periphery of the roll of paper reaches a first outer diameter smaller than the predetermined diameter;
a second sensor configured to detect whether the outer periphery of the roll of paper reaches a second outer diameter smaller than the first outer diameter; and
an estimation unit configured to estimate a remaining amount of paper in the roll of paper based on a thickness of the paper, wherein the thickness of the paper is estimated based on a length of paper discharged during a time interval between a point of time when the first sensor detects the outer periphery of the roll of paper at the first outer diameter and a point of time when the second sensor detects the outer periphery of the roll of paper at the second outer diameter.

16. The printer of claim 15, wherein the estimation unit is further configured to estimate the remaining amount of paper in the roll of paper by the thickness of the paper, the second outer diameter and a diameter of a core.

17. The printer of claim 15, wherein the length of the paper discharged is estimated based on the number of driving pulses generated by a platen motor configured to drive a platen to discharge the paper, wherein the number of driving pulses is counted from the time when the first sensor detects the outer periphery of the roll of paper at the first outer diameter until the time when the second sensor detects the outer periphery of the roll of paper at the second outer diameter.

18. The printer of claim 15, wherein, when N indicates the center of the roll of paper with a maximum outer diameter, a first line is defined by connecting between the center N and the center of the roll of paper when contained in the first recessed portion, and a second line is defined by connecting between the center N and the center of the roll of paper when contained in the second recessed portion, the first and second lines form a predetermined angle therebewteen,
wherein the first and second sensors are aligned in a line with respect to the center N at about half of the predetermined angle.
